(19) United States
(12) Patent Application Publication
(10) Pub. No.: US 2020/0214874 A1
(43) Pub. Date: Jul. 9, 2020

(54) GAUNTLET MOTIVE BATTERY

(71) Applicant: Joe Ernest BROWN, Grass Valley, CA (US)

(72) Inventor: Joe Ernest BROWN, Grass Valley, CA (US)

(21) Appl. No.: 16/826,308

(22) Filed: Mar. 23, 2020

Related U.S. Application Data
(63) Continuation of application No. 16/071,044, filed on Jul. 18, 2018.

(51) Int. Cl. A61F 5/445; A61F 5/44

(52) U.S. Cl. CPC A61F 5/445; A61F 5/4407

(57) ABSTRACT

An improved gauntlet wet cell battery provides a plurality of elongated hollow spines filled with active material in a cathode array. Bottom ends of the cathode array are closed with an electrically conductive bottom end cap that electrically and mechanically interconnects the free ends of each spine so as to close and substantially rigidly locate the free ends of said spines with respect to one another. In this manner, electrical continuity between the spines is maintained even if one of the spines fractures, cracks, or otherwise becomes mechanically disassociated from an upper portion of the same spine.

### TECHNICAL FIELD

The invention relates to wet cell batteries. More specifically, the invention relates to an improvement in gauntlet lead-acid wet cell batteries.

### BACKGROUND OF THE INVENTION

Gaston Plante was a French physicist who is generally acknowledged to have invented the lead-acid battery in 1859. The lead-acid battery eventually became the first commercial rechargeable electric battery. His early model consisted of two sheets of coded lead soaked in sulfuric acid. In the following year he presented a 9-cell lead-acid battery to the French Academy of Sciences. In 1881, Camille (Emile Alfonse) Faure would develop a more efficient and reliable model that saw success in early electric cars. Faure's improvement included a process for making lead paste to “fill in” what has today become a lead grid, providing the plate with tremendous surface area for use with both a positive and negative plate in a lead-acid battery.

Since that time over 100 years ago, there have been numerous improvements in lead-acid battery technology with respect to the mechanical structure of such batteries or, “wet cells.” However, the basic electrochemistry of how the battery is formed, charged and maintained after the battery is manufactured has not changed substantially.

With reference to FIG. 1 of the drawings, those of ordinary skill in the art will appreciate that a conventional lead-acid battery generally indicated at reference numeral 10 in FIG. 1 consists of a fluid-impermeable case 12 containing an electrolyte in solution 14, typically a dilute mixture of sulfuric acid (H₂ SO4) and water (H₂O). The sulfuric acid disassociates in water to form sulfate anions (SO₄^{2-aq}) and hydrogen cations (H⁺). An anode plate 16 consists primarily of lead (Pb) and is selectively electrically communicated through a load 18 to a cathode 20, typically a mesh grid structure coated with a lead peroxide paste (PbO₂). In a commercial battery, an ion-porous separator 19 is interposed between the anode and cathode plates 16, 20 to prevent them from coming into mechanical contact with one another, thus creating an electrical short. The anode and cathode are selectively placed in electrical communication through the load 18 by a switch 22. To discharge the battery or wet cell 10 through the load 18, the switch 22 is closed causing chemical reactions to occur at both the anode 16 and cathode 20. During discharge, lead from the anode 16 combines with aqueous sulfite anions to form lead sulfate in solid form liberating two electrons. This reaction can be chemically described as follows:

Pb(s)+SO₄²⁻(AQ)→PbSO₄(s)+2e⁻ (1)

The electrons travel through the switch 22 and load 18 into the cathode 20 where lead peroxide in solid form combines with aqueous sulfate anions and four hydrogen cations, including the two electrons that were liberated from the anode forming lead sulfate on the surface of the cathode and two water molecules. This reaction can be chemically described as follows:

PbO₂(s)+SO₄²⁻(AQ)+4H⁺+2e⁻→PbSO⁴(s)+2H₂O(l) (2)

As is well known to those of ordinary skill in the art, to recharge the battery the load may be removed and a reverse polarity applied to the cathode and anode such that the above chemical reactions are reversed. Care must be taken to prevent overcharging the battery, which will cause the water in the electrolyte solution to boil, exposing the anode and cathode. If portions of the anode and cathode are exposed during discharge, adverse mechanical reactions will occur to the plates.

An early improvement to the standard Faure lead plate battery was the development of the so-called gauntlet tubular plate battery, developed by Exide, USA in 1908. The gauntlet battery consists of a series of standard flat anode plates. Instead of flat plates for the cathode, the cathode plates are replaced with a series of tubular arrays consisting of vertically oriented conductive spines surrounded by a fabric sleeve or gauntlet. An interstitial annular void between the gauntlet and the conductive spine is filled with an active lead, such as lead oxide. The top of each spine in a gauntlet plate is interconnected with a lead bar so that the spines are electrically interconnected with one another. The tubular gauntlet plates are then interposed between adjacent flat planar anode plates of the conventional variety. A variety of advanced techniques have been developed for creating the gauntlets themselves, (see U.S. Pat. No. 4,048,399, issued to Terzaghi, on Sep. 3, 1997) as well as means for filling the interstitial areas between the lead spines and the gauntlet itself with active material, as set forth in U.S. Pat. No. 3,945,097 to Daniels, Jr. et al., issued Mar. 23, 1976. The disclosures of the above-listed patents are herein incorporated by reference in their entirety for purposes of a complete disclosure. See also U.S. Pat. No. 5,134,045 to Lanari issued Jul. 28, 1992. The gauntlet battery is superior to the standard plate-type battery in terms of energy density and resistance of the cathode spines from mechanical degradation due to the supporting structure of the gauntlet itself. The gauntlet material is typically a porous fiber that is strengthened with an ion-permeable resin. Several different forms of materials have been used to create the gauntlets themselves but, in 1973, all such materials were substantially replaced with polyester yarn. The polyester fabric gauntlets in particular advantageously preclude any sheded active material from the gauntlet spines from finding its way to the bottom of the battery case and possibly creating an undesirable short circuit between the cathode spines and an adjacent anode plate. In order to mechanically maintain the free ends of the spines in spaced relationship to one another and to prevent mechanical shocks, the ends of the spines are typically capped with a nonconductive plastic end piece such that the ends of the spines maintain their spatial relationship with respect to one another.

One disadvantage of the modern gauntlet battery described above is that the spines themselves are somewhat fragile; thus, a mechanical shock to the battery may result in one of the spine's cracking and losing electrical continuity with that portion of the spine above the break and, hence, the rest of the spines within the same gauntlet cathode plate. The gauntlet fabric will nevertheless typically hold the broken lower end of the spine substantially in place so as to not create a short circuit with any adjacent anode plates; however, the electrical charge storage capacity of the broken spine is forever lost from the battery.

It is therefore an object of the present invention to provide an improved gauntlet wet cell battery in which a fractured spine remains in electrical communication with the remainder of the gauntlet spine cathode plate.

### BRIEF SUMMARY OF THE INVENTION

It is therefore an object of the present invention to provide an improved gauntlet wet cell battery in which a broken spine within a gauntlet cathode plate remains in electrical continuity with the remainder of the gauntlet plate.

The invention achieves this object, and other objects and advantages of the invention that will become apparent from the description that follows, by providing a substantially fluid-impervious battery case including a conventional anode plate and an improved gauntlet cathode array having a plurality of hollow elongated spines filled with an active material. Each spine of the array defines a top end and a distal free end wherein the spines are electrically and mechanically interconnected at the top ends by an integral conductive structure. The improved gauntlet cathode array includes a substantially conductive bottom end cap electrically and mechanically interconnected with the spine free ends so as to electrically close and substantially rigidly locate the free ends with respect to one another. Preferably, a plurality of ion-permeable fabric covers substantially encase the elongated spines, and an electrolyte in solution is provided with water in the battery case such that the anode plate, the gauntlet cathode array, the bottom end cap, and the fabric covers are all substantially received in the battery case.

In the preferred embodiment of the invention the hollow spines consist of an elongated, central lead alloy member surrounded by a fabric gauntlet so as to present an elongated tubular or annular interstitial space filled with an active material, such as lead oxide. The preferred battery includes one more anode plate than cathode array in each battery.

In an alternate embodiment, the anode plate is also an array including a plurality of elongated hollow spines filled with an active material, such as lead oxide. In this alternate embodiment, each spine of the array defines a top end and distal free wherein the spines are electrically and mechanically interconnected to the top ends by an integral structure. The bottom ends are also preferably electrically and mechanically interconnected by an electrically conductive bottom end cap. In alternate embodiments of the invention, the active material in the spines is substantially powdered lead and the fabric covers that comprise the gauntlets are substantially manufactured from one of the following group of materials: carbon fiber; polyester fiber; or Kevlar® fiber. In the improved battery, the electrolyte essentially consists of an Oxonium based electrolyte, preferably H₉O₄ and the bottom end caps are substantially manufactured from lead or a conventional lead alloy. Finally, in a battery having a single gauntlet cathode array, the battery case itself may be manufactured from lead and comprise the anode plate.

### BRIEF DESCRIPTION OF THE DRAWINGS

FIG. 1 is a schematic representation of a prior art wet cell battery and related chemical reactions.

FIG. 2 is a partial, exploded isometric perspective view of an improved gauntlet wet cell battery of the present invention, showing two single gauntlet cathode arrays and a single exemplary anode plate.

FIG. 3 is an enlarged, perspective isometric view of free ends of the gauntlet cathode array of the present invention, including an electrically conductive end cap therefor.

FIG. 4 is a perspective view of an alternate embodiment of the invention in which both the anode plate and the cathode plate consist of a series of gauntlet plates in a wet cell battery.

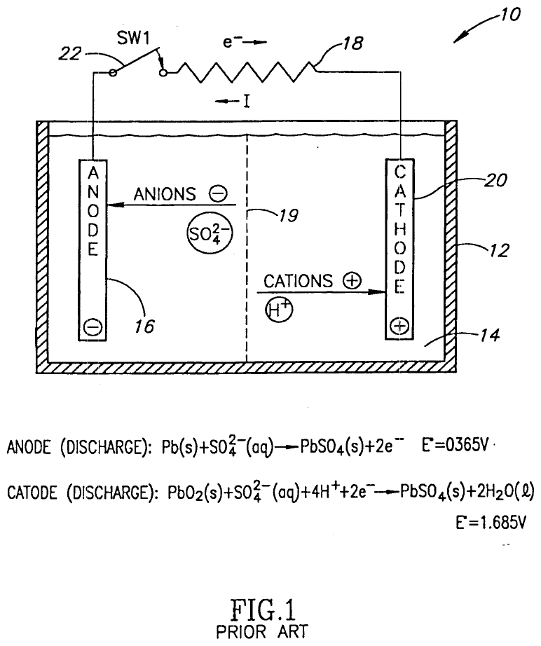

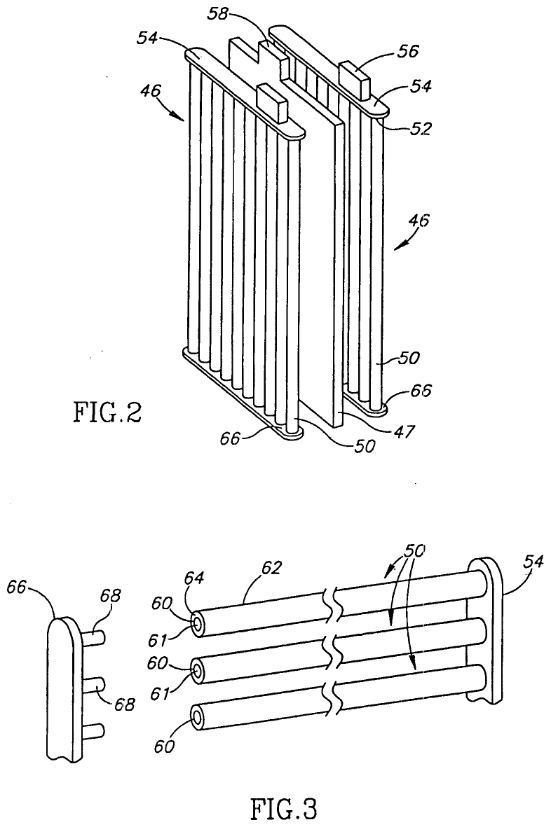

### DETAILED DESCRIPTION OF THE PREFERRED EMBODIMENTS

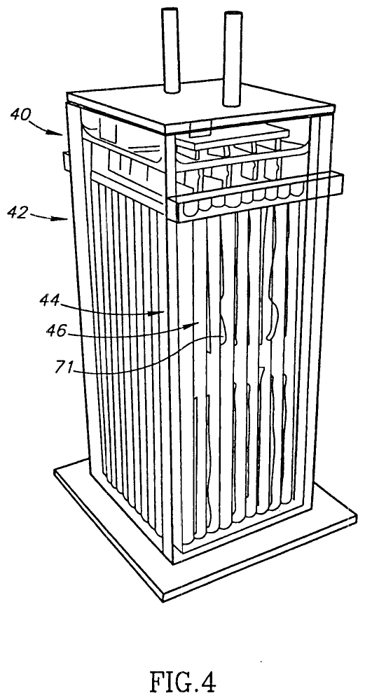

An improved gauntlet motive battery or improved gauntlet wet cell battery is generally indicated at reference numeral 40 in FIG. 4. The battery consists of a fluid-impervious battery case 42 containing a series of anode and cathode plates 44, 46. In the preferred embodiment of the invention, at least the cathode plate 46 consists of a series of hollow, elongated spines 50 preferably manufactured from lead or lead alloyed with tin, antimony, calcium, selenium or another metal to provide mechanical strength to the spine. The top ends 52 of each spine are interconnected by atop end cap 54 so as to form an integral structure with the spines. The top end cap may include a conventional tab 56 for interconnecting the cathode arrays electrically with one another. The anode plate 44 is of the conventional type and also provided with a tab 28 for a similar purpose.

As best seen in FIG. 3, each spine 50 consists of a solid, elongated central conductor 60 manufactured from the same material as the top end cap 54, preferably lead or lead alloy. Each conductor 60 is surrounded by a radially spaced-apart gauntlet 62 preferably consisting of a fabric material, which is carbon fiber, polyester fiber, or Kevlar® fiber stiffened with an appropriate ion-permeable epoxy-like material or resin, as is well known to those of ordinary skill in the art. The conductor 60 and gauntlet 62 thus form an elongated annular tube 64 that is preferably filled with an active material such as lead oxide or powdered lead. The active material receives charge during the charging process and releases electrons during the discharge process.

In contrast to prior art gauntlet batteries, the cathode arrays 46 are provided with bottom end caps 66 made from a conductive material, preferably lead or lead alloy. The bottom end caps 66 are provided with annular nipples 68 spaced along the end caps in series so as to register with the central conductors 60 associated with the spines 50 of each cathode array. The nipples 68 of the end caps 66 preferably define apertures to receive ends of the central conductors 60 so that the end caps may be soldered or the like to the central conductors 60. A thermal barrier (or insulator-not shown) such as plastic or leather may be interposed between the end caps 66 and the fabric gauntlets 62 to prevent the polymeric fabric from melting when the end caps 66 are soldered to the central conductors 60. The nipples are also adapted so as to be received in the annular depression at the end of each annular tube 64 so as to mechanically and electrically interconnect the free ends 61 of each conductor 60. In this way, any spine 50 that is fractured will maintain electrical continuity with an adjacent spine through the nipple 68 and end cap 66 so as to provide full electrical access to the conductor 30 and active material received in the annular tube 64 for charging and discharging purposes. One embodiment of the invention shown in FIG. 4 includes the improved gauntlet wet cell battery 40 disclosed above, wherein the anode plates 44 are also gauntlet arrays of the type described above. In this battery, the anode and cathode plates can be placed in close register with one another such that each cathode and anode spine is partially offset so as to fill voids there between, resulting in a very compact battery structure. As with any conventional wet cell battery, the anode and cathode plates, whether tubular or plate-like, may be separated by a nonconductive ion porous separator 71 or the like in the conventional manner.

Before a lead-acid battery can be used it must be electro-chemically “formed.” During forming, the active material on the cathode plate withdraws the sulfuric acid which was used in the lead paste manufacturing process. The dissolving and removing of the sulfuric acid allows the lead oxide molecules to become interstitial to the act of coating. The invention battery 40 may be formed with a very low specific gravity sulfuric electrolyte for this forming process and when the action is complete will have an electrolyte of standard 1.280 specific gravity. Alternatively, the battery may be formed with hundreds of plates at the same time in large tanks. These plates are then washed with water and dried in ovens. After they are manufactured into batteries they are called “dry formed” batteries. These batteries are shipped to dealers in their dry state and sulfuric acid electrolyte of 1.280 standard gravity is added for sale.

The improved gauntlet battery 40 has particular applicability where a high energy density to weight ratio is desired (e.g., motive battery applications). However, the battery also has utility in the solar, recreational vehicle, cell tower and military applications.

Those of ordinary skill in the art will conceive of other alternate embodiments of the invention upon reviewing this disclosure. Thus, the invention is riot to be limited to the above description, but is to be determined in scope by the claims, which follow.

I claim:

1. An improved gauntlet wet cell battery, comprising:
a substantially fluid-impervious battery case;
an anode plate;
a gauntlet cathode array having a plurality of elongated hollow spines filled with an active material, each spine of the array defining a top end and a distal free end, the spines being electrically and mechanically interconnected at the top ends by an integral structure;
a substantially conductive bottom end cap electrically and mechanically interconnected with the spine free ends so as to close and substantially rigidly locate the free ends with respect to one another;
a plurality of ion-permeable fabric covers substantially encasing the elongated spines; and
an electrolyte in solution with water in the battery case such that the anode plate, gauntlet cathode array, bottom end cap, and fabric covers are all substantially received in the battery case.

2. The improved gauntlet wet cell battery of claim 1, including an ion-porous separator between the anode plate and cathode array.

3. The improved gauntlet wet cell battery of claim 1, wherein the anode plate is also an array having a plurality of elongated hollow spines filled with an active material, each spine of the array defining a top end and a distal free end, the spines being electrically and mechanically interconnected at the top and bottom ends by an integral conductive structure.

4. The improved gauntlet wet cell battery of claim 3, wherein a number of cathode spines is one less than a number of anode spines and the spines are closely packed together.

5. The improved gauntlet wet cell battery of claim 1, wherein the anode plate is manufactured from a material selected from one of the following: foamed lead, compressed lead wool, lead sheets and composite material.

6. The improved gauntlet wet cell battery of claim 1, wherein the active material in the spines is substantially powdered lead.

7. The improved gauntlet wet cell battery of claim 1, wherein the fabric covers are substantially manufactured from one of a group consisting of carbon fiber, polyester fabric, and Kevlar® fiber.

8. The improved gauntlet wet cell battery of claim 1, wherein the electrolyte consists essentially of H₉O₄.

9. The improved gauntlet wet cell battery of claim 1, wherein the bottom end cap is substantially manufactured from lead.

10. The improved gauntlet wet cell battery of claim I, wherein the anode plate is the battery case.

11. The improved gauntlet wet cell battery of claim 1, wherein each spine consists of a central elongated conductor substantially surrounded by an elongated annulus consisting of active material radially restrained by a fabric gauntlet coated with an ion permeable resin.
